(19) United States
(12) Patent Application Publication
(10) Pub. No.: US 2022/0284817 A1
(43) Pub. Date: Sep. 8, 2022

(54) INFECTION CONTROL SYSTEM AND INFECTION CONTROL METHOD

(71) Applicant: TOYOTA JIDOSHA KABUSHIKI KAISHA, Toyota-shi Aichi-ken (JP)

(72) Inventors: Miki NOMOTO, Nisshi-shi Aichi-ken (JP); Taizo Masuda, Yokohama-shi Kanagawa-ken (JP); Yuta Kataoka, Susono-shi Shizuoka-ken (JP); Hiromitsu Kobayashi, Nisshini-shi Aichi-ken (JP); Yoshiki Ueda, Susono-shi Shizuoka-ken (JP); Satoshi Omi, Ebina-shi Kanagawa-ken (JP); Yuki Nishikawa, Susono-shi Shizuoka-ken (JP)

(21) Appl. No.: 17/564,735

(22) Filed: Dec. 29, 2021

(30) Foreign Application Priority Data
Mar. 2, 2021 (JP) 2021-032454

(51) Int. Cl. G08G 1/00; A61B 5/01; A61B 5/08; A61B 5/00; G06Q 10/02; G06Q 50/30

(52) U.S. Cl. CPC G08G 1/202; A61B 5/01; A61B 5/0823; A61B 5/74; G06Q 10/02; G06Q 50/30

(57) ABSTRACT

An infectious disease detection sensor for detecting whether a waiting passenger of a bus is suspected to be infected with an infectious disease is installed at a bus stop, and whether the waiting passenger of the bus is a suspected infected person who is suspected of being infected with the infectious disease is determined based on a detection result detected by the infectious disease detection sensor. When the waiting passenger of the bus is determined to be the suspected infected person, boarding of the suspected infected person to the bus is refused, and a dedicated vehicle for the suspected infected person is dispatched to the bus stop separately from the bus.

### CROSS-REFERENCE TO RELATED APPLICATION

This application claims priority to Japanese Patent Application No. 2021-032454 filed on Mar. 2, 2021, incorporated herein by reference in its entirety.

### BACKGROUND

### 1. Technical Field

The present disclosure relates to an infection control system and an infection control method.

### 2. Description of Related Art

A seat device in which an infrared sensor capable of detecting a body temperature of a passenger is installed on a seat of mass transportation means of passengers, for example, an aircraft to determine whether the passenger is infected with an infectious disease involving fever based on the body temperature of the passenger detected by the infrared sensor is known (for example, refer to Japanese Unexamined Patent Application Publication No. 2013-39878 (JP 2013-39878 A)).

### SUMMARY

However, with the seat device, for example, there is an issue that, since the body temperature of the passenger who is already on board the aircraft is detected, when the passenger is determined to be infected with an infectious disease, the infectious disease has already spread to other passengers.

Therefore, the present disclosure provides an infection control system including a vehicle dispatch coordination department that dispatches a dedicated vehicle for a suspected infected person separately from a bus when an infectious disease determination device that is installed at a bus stop and that determines whether a waiting passenger of the bus is the suspected infected person who is suspected of being infected with an infectious disease determines that the waiting passenger of the bus is the suspected infected person. Further, the present disclosure provides an infection control method including dispatching a dedicated vehicle for a suspected infected person separately from a bus when an infectious disease determination device that is installed at a bus stop and that determines whether a waiting passenger of the bus is the suspected infected person who is suspected of being infected with an infectious disease determines that the waiting passenger of the bus is the suspected infected person.

This can suppress the waiting passengers of the bus other than the suspected infected person from being infected with the infectious disease in the bus.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram showing a vehicle and a server graphically represented;

FIG. 2 is a functional configuration diagram for causing a bus to autonomously travel;

FIG. 3 is a flowchart for controlling an operation of the bus;

FIG. 4 is a diagram showing the entire infection control system;

FIG. 5 is a diagram showing an infectious disease determination device installed at a bus stop;

FIG. 6 is a diagram showing a vehicle dispatch coordination department;

FIG. 7 is a flowchart for making a boarding request;

FIG. 8 is a flowchart for executing bus reception processing;

FIG. 9 is a flowchart for executing boarding processing; and

FIG. 10 is a flowchart for executing vehicle dispatch processing.

### DETAILED DESCRIPTION OF EMBODIMENTS

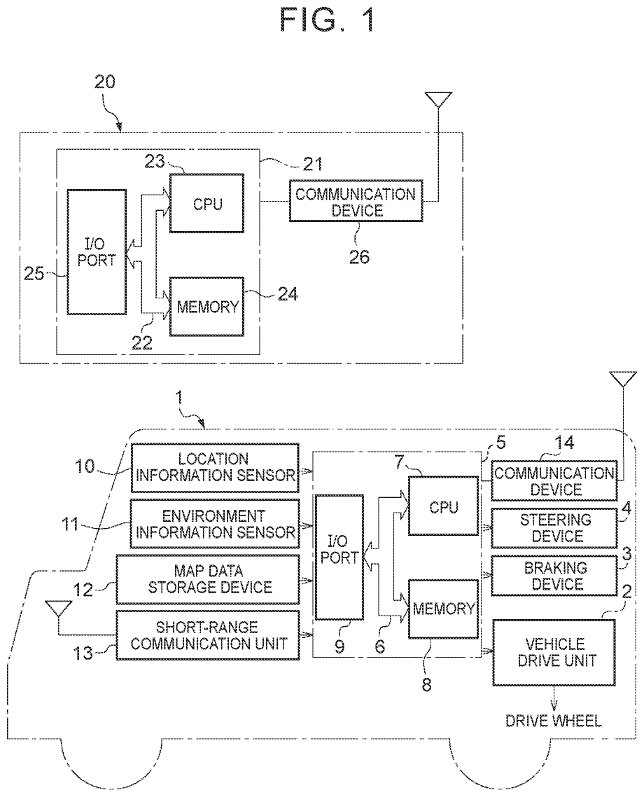

With reference to FIG. 1, the numeral 1 illustrates the bus graphically. In an embodiment according to the present disclosure, a bus 1 is a circulation bus that can be caused to autonomously travel along a preset traveling route within a preset region. Further, in FIG. 1, the numeral 2 indicates a vehicle drive unit for applying a driving force to drive wheels of the bus 1, the numeral 3 indicates a braking device for braking the bus 1, the numeral 4 indicates a steering device for steering the bus 1, and the numeral 5 indicates an electronic control unit mounted in the bus 1. As shown in FIG. 1, the electronic control unit 5 is composed of a digital computer, and includes a central processing unit (CPU: microprocessor) 7, a memory 8 composed of a read-only memory (ROM) and a random access memory (RAM), and an input/output port 9 that are connected to each other by a bidirectional bus 6.

Further, as shown in FIG. 1, the bus 1 is provided with a location information sensor 10, an environment information sensor 11, a map data storage device 12, and a short-range communication unit 13. The location information sensor 10 is a sensor for detecting the current location of the bus 1. The location information sensor 10 is composed of, for example, a global positioning system (GPS) that receives a radiowave from an artificial satellite and detects the current location of the bus 1. Further, the environment information sensor 11 includes a sensor that detects the state of the bus 1 and a sensor that detects the periphery of the bus 1 to cause the bus 1 to autonomously travel. In this case, an acceleration sensor, a speed sensor, and an azimuth angle sensor are used as the sensor that detects the state of the bus 1, and a camera for capturing images of the front of the bus 1 or the like, light detection and ranging (LIDAR), and a radar are used as the sensor that detects the periphery of the bus 1.

Further, the map data storage device 12 stores map data and the like that are necessary for causing the bus 1 to autonomously travel. Further, the short-range communication unit 13 is provided for reading information from a mobile terminal of a person who boards the bus 1 using, for example, a radio frequency identification (DFID) system. For example, an entrance of the bus 1 is provided with an information reading unit that makes a beeping sound when the mobile terminal is brought close to the information reading unit and reads the information of the mobile terminal. The short-range communication unit 13 executes this reading of the information of the mobile terminal. The location information sensor 10, the environment information sensor 11, the map data storage device 12, and the short-range communication unit 13 are connected to the electronic control unit 5.

Further, in FIG. 1, the numeral 20 indicates a server. As shown in FIG. 1, an electronic control unit 21 is installed in the server 20. The electronic control unit 21 is composed of a digital computer, and includes a CPU (microprocessor) 23, a memory 24 composed of a ROM and a RAM, and an input/output port 25 that are connected to each other by a bidirectional bus 22. Further, a communication device 26 for communicating with the bus 1 and the like is installed in the server 20. The bus 1 is equipped with a communication device 14 for communicating with the server 20 and the like.

In the embodiment according to the present disclosure, the vehicle drive unit 2 is composed of an electric motor driven by a secondary battery or an electric motor driven by a fuel cell. Driving of the drive wheels is controlled by the electric motor as described above in accordance with an output signal from the electronic control unit 5. Further, the braking control of the bus 1 is executed by the braking device 3 in accordance with the output signal from the electronic control unit 5. The steering control of the bus 1 is executed by the steering device 4 in accordance with the output signal from the electronic control unit 5.

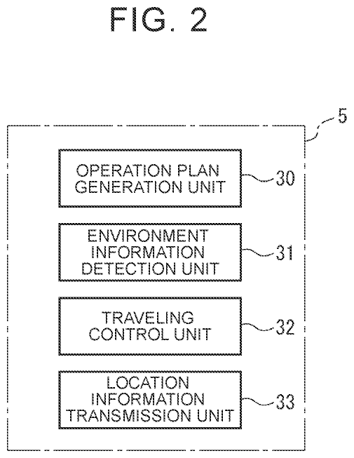

FIG. 2 shows a functional configuration diagram for causing the bus 1 to autonomously travel. As shown in FIG. 2, in the embodiment according to the present disclosure, the bus 1 includes an operation plan generation unit 30, an environment information detection unit 31, a traveling control unit 32, and a location information transmission unit 33. When the operation plan generation unit 30 receives an operation command from the server 20, the operation plan generation unit 30 generates an operation plan such as a traveling route, a traveling speed, and a stop position of the bus 1 based on the operation command. In the environment information detection unit 31, the environment information sensor 11 detects the environmental information necessary for the autonomous traveling of the bus 1. For example, in the environment information detection unit 31, the number and positions of lanes, the number and positions of other moving objects present around the bus 1, and the number and positions of obstacles (for example, pedestrians, bicycles, structures, buildings) present around the bus 1, the structure of the roads, and road signs are detected.

The traveling control unit 32 executes the traveling control for causing the bus 1 to autonomously travel on the operation plan generated by the operation plan generation unit 30, the environment information detected by the environment information detection unit 31, the map data stored in the map data storage device 12, and the current location of the bus 1 detected by the location information sensor 10. With this configuration, the bus 1 can be caused to autonomously travel along the set traveling route while avoiding contact with other moving objects and obstacles. The location information transmission unit 33 transmits information relating to the current location of the bus 1 detected by the location information sensor 10 to the server 20 via the communication device 14. Note that, the operation plan generation unit 30, the environment information detection unit 31, the traveling control unit 32, and the location information transmission unit 33 are provided in the electronic control unit 5 of the bus 1.

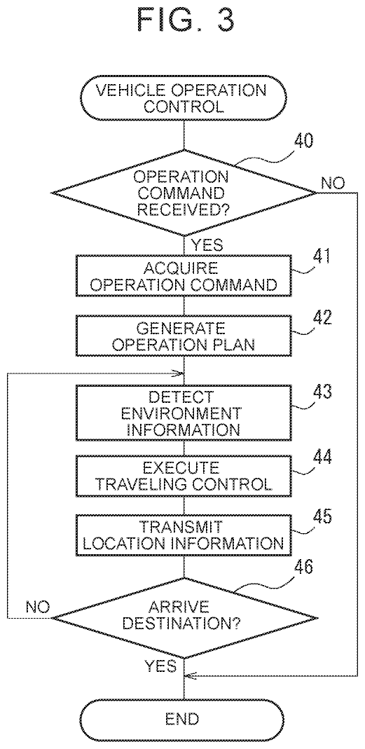

FIG. 3 shows a vehicle operation control routine executed by the electronic control unit 5 mounted on the bus 1 to cause the bus 1 to autonomously travel. This routine is executed by interrupts at regular intervals. With reference to FIG. 3, first, in step 40, the electronic control unit 5 determines whether an operation command has been received from the server 20. When the electronic control unit 5 determines that the operation command has not been received from the server 20, the processing cycle is terminated. On the other hand, when the electronic control unit 5 determines that the operation command has been received from the server 20, the process proceeds to step 41 and the operation command is acquired. Next, in step 42, the operation plan such as the traveling route, the traveling speed, and the stop position of the bus 1 is generated based on the operation command.

Next, in step 43, the environment information necessary for autonomous traveling of the bus 1 is detected. Next, in step 44, the traveling control for causing the bus 1 to autonomously travel is executed, whereby the bus 1 can be caused to autonomously travel along the set traveling route while avoiding contact with other moving objects and obstacles. Then, in step 45, information relating to the current location of the bus 1 is transmitted to the server 20. Next, in step 46, the electronic control unit 5 determines whether the bus 1 has arrived the destination set by the operation command. In this case, in the embodiment according to the present disclosure, the electronic control unit 5 determines whether the bus 1 has arrived the set destination after circulating a set number of times. When the electronic control unit 5 determines that the bus 1 has not arrived the destination after circulating the set number of times, the process returns to step 43, and the autonomous traveling control of the bus 1 is continued. When the electronic control unit 5 determines that the bus 1 has arrived the destination after circulating the set number of times, the processing cycle is terminated.

When a person infected with the infectious disease boards the bus 1 while the bus 1 is operated as described above, there arises an issue that another person who is already in the bus is infected. Therefore, in the embodiment according to the present disclosure, an infectious disease determination device for determining whether a waiting passenger of the bus is suspected of being infected with an infectious disease is installed at a bus stop, and a dedicated bus for a suspected infected person is dispatched to the bus stop separately from the bus when the waiting passenger of the bus is determined to be the suspected infected person.

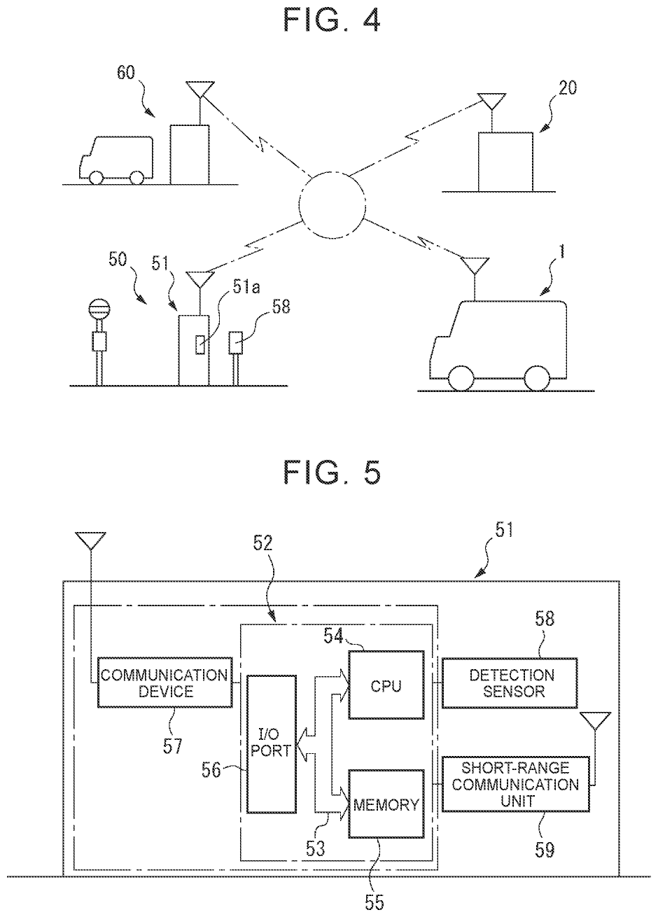

FIG. 4 shows the entire infection control system according to the present disclosure. With reference to FIG. 4, in addition to the bus 1 and the server 20 shown in FIG. 1, a bus stop 50 and a vehicle dispatch coordination department 60 are graphically shown in FIG. 4. As shown in FIG. 4, the bus stop 50 is equipped with an infectious disease determination device 51 for determining whether the waiting passenger of the bus 1 is suspected of being infected with the infectious disease. The infectious disease determination device 51 is shown in FIG. 5. Referring to FIG. 5, an electronic control unit 52 is provided in the infectious disease determination device 51. The electronic control unit 52 is composed of a digital computer, and includes a CPU (microprocessor) 54, a memory 55 composed of a ROM and a RAM, and an input/output port 56 that are connected to each other by a bidirectional bus 53. A communication device 57 for communicating with the bus 1, the server 20, and the vehicle dispatch coordination department 60 is connected to the electronic control unit 52.

Further, as shown in FIG. 5, an infectious disease detection sensor 58 for detecting whether the waiting passenger of the bus 1 is suspected of being infected with the infectious disease is connected to the electronic control unit 52. FIG. 4 graphically shows the infectious disease detection sensor 58 installed at the bus stop 50. As the infectious disease detection sensor 58, various types of sensors such as a thermo-sensor capable of detecting the body temperature in a non-contact state, a contact-type body temperature sensor capable of detecting the body temperature, a microphone capable of detecting a coughing sound, and a chemical sensor capable of chemically detecting symptoms of the infectious disease can be used.

Further, as shown in FIG. 5, a short-range communication unit 59 similar to the short-range communication unit 13 shown in FIG. 1 is connected to the electronic control unit 52. For example, the infectious disease determination device 51 is provided with an information reading unit 51a that makes a beeping sound when the mobile terminal is brought close to the information reading unit and reads the information of the mobile terminal. The short-range communication unit 59 executes this reading of the information of the mobile terminal at the information reading unit 51a. FIG. 4 graphically shows the information reading unit 51a.

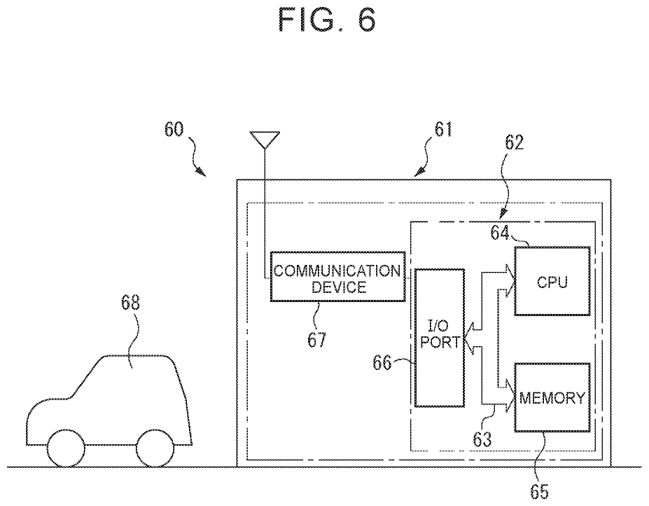

FIG. 6 shows the vehicle dispatch coordination department 60 shown in FIG. 4. As shown in FIG. 6, a vehicle dispatch coordination device 61 is installed in the vehicle dispatch coordination department 60, and an electronic control unit 62 is provided in vehicle dispatch coordination device 61. The electronic control unit 62 is composed of a digital computer, and includes a CPU (microprocessor) 64, a memory 65 composed of a ROM and a RAM, and an input/output port 66 that are connected to each other by a bidirectional bus 63. A communication device 67 for communicating with the bus 1, the server 20, and the infectious disease determination device 51 is connected to the electronic control unit 62. Further, as shown in FIG. 6, in the vehicle dispatch coordination department 60, a plurality of dedicated vehicles 68 for the suspected infected person are made to stand by in order to transport the person suspected of being infected with the infectious disease upon request.

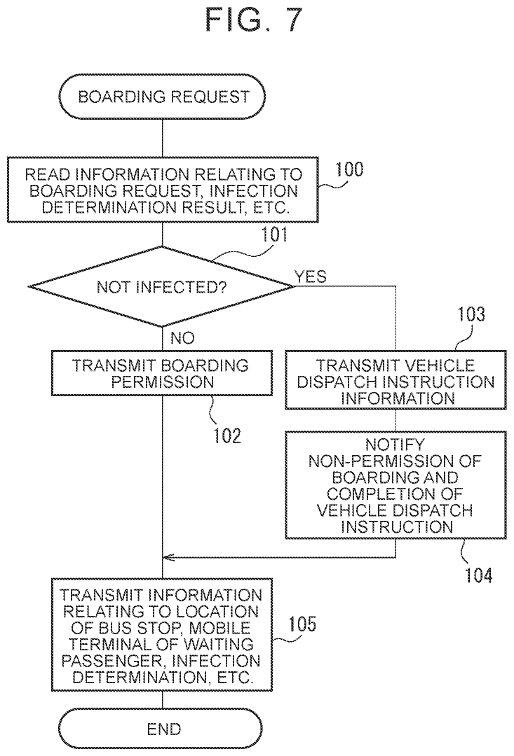

Next, an example of the infection control system according to the present disclosure will be described with reference to FIGS. 7 to 10. It should be noted that FIGS. 7 to 10 describe the infection control system according to the present disclosure by taking the case where the boarding permit of the bus 1 is read into the mobile terminal owned by the person who boards the bus 1 as an example. However, this is only an example, and various methods other than the method using the mobile terminal can be adopted. First, a method of boarding the bus 1 will be described with reference to a boarding request routine shown in FIG. 7 executed in the electronic control unit 52 of the infectious disease determination device 51 installed at the bus stop 50.

In the example shown in FIG. 7, when the person who intends to board the bus 1 arrives at the bus stop 50, the person who intends to board the bus 1, that is, the waiting passenger of the bus 1, first receives a detection diagnosis by the infectious disease detection sensor 58 so as to determine whether the waiting passenger is suspected of being infected with the infectious disease, and reads the determination result based on the detection diagnosis by the infectious disease detection sensor 58 into the mobile terminal of the waiting passenger. Next, the information of the mobile terminal owned by the waiting passenger is read by the information reading unit 51a of the infectious disease determination device 51. At this time, as shown in step 100 of the boarding request routine shown in FIG. 7, the information indicating that the boarding request is being made, the determination result by the infectious disease detection sensor 58, and the information relating to the mobile terminal of the waiting passenger, for example, the owner information of the mobile terminal, are read by the information reading unit 51a.

Then, in step 101, whether the waiting passenger of the bus is infected with the infectious disease is determined based on the information read in step 100. When the waiting passenger of the bus is determined to be not infected with the infectious disease, a message is transmitted to the mobile terminal of the waiting passenger via the communication network to permit the waiting passenger to board the bus 1 in step 102. Next, in step 105, information related to the location of the bus stop 50 where the waiting passenger is present, information relating to the mobile terminal of the waiting passenger, for example, the owner information of the mobile terminal, information relating to the determination result by the infectious disease detection sensor 58, and the like are transmitted to the bus 1 via the server 20 or directly.

On the other hand, when the waiting passenger of the bus is determined to be infected with the infectious disease in step 101, the process proceeds to step 103, and vehicle dispatch instruction information, that is, an instruction to dispatch a vehicle to the bus stop 50 where the waiting passenger is present, the information relating the location of the bus stop 50 where the waiting passenger is present, the information relating to the mobile terminal of the waiting passenger, for example, the owner information of the mobile terminal, the information relating to the determination result by the infectious disease detection sensor 58 are transmitted to the vehicle dispatch coordination department 60. Next, in step 104, a message that the waiting passenger is not permitted to board the bus 1 and the vehicle is already dispatched is transmitted to the mobile terminal of the waiting passenger via the communication network. Then, the process proceeds to step 105.

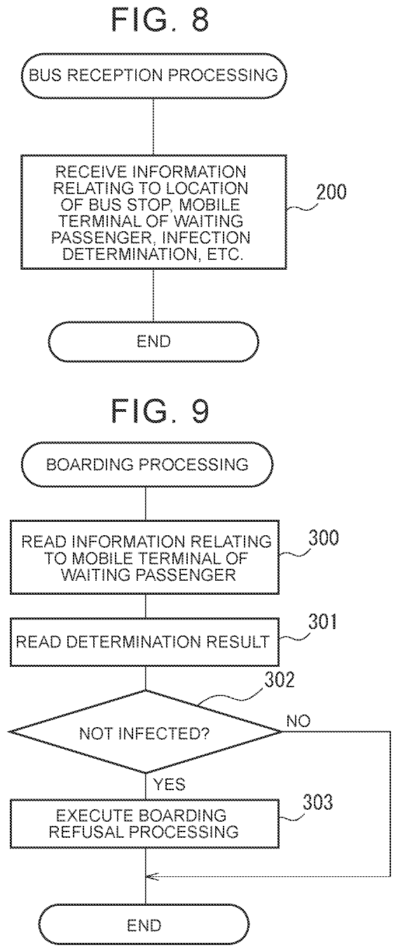

FIG. 8 shows a reception processing routine executed by the electronic control unit 5 of the bus 1 in operation. With reference to FIG. 8, in step 200, the information transmitted in step 105 in FIG. 7, that is, the information relating to the location of the bus stop 50 where the waiting passenger is present, the information relating to the mobile terminal of the waiting passenger, for example, the owner information of the mobile terminal, the information relating to the determination result by the infectious disease detection sensor 58, and the like are received.

FIG. 9 shows a boarding processing routine executed by the electronic control unit 5 of the bus 1 when the waiting passenger boards the bus 1 at the bus stop 50. Note that, as described above, the entrance of the bus 1 is provided with an information reading unit that makes a beeping sound when the mobile terminal is brought close to the information reading unit and reads the information of the mobile terminal. When the waiting passenger boards the bus 1, the waiting passenger performs an operation to read the information of the mobile terminal of the waiting passenger by the information reading unit. At this time, as shown in step 300 of the boarding processing routine in FIG. 9, information relating to the mobile terminal of the waiting passenger, for example, the owner information of the mobile terminal, is read. Next, in step 301, the electronic control unit 5 determines whether the waiting passenger who is about to board the bus 1 is infected with the infectious disease based on the information that has been already transmitted to the bus 1. The processing cycle is terminated when the electronic control unit 5 determines that the waiting passenger of the bus is not infected with the infectious disease. On the other hand, when the electronic control unit 5 determines in step 302 that the waiting passenger of the bus is infected with the infectious disease, the process proceeds to step 303 and boarding refusal processing is executed.

Next, this boarding refusal processing will be described. As described above, when the electronic control unit 5 determines that the waiting passenger of the bus is infected with the infectious disease, a message that the waiting passenger is not permitted to board the bus 1 is transmitted to the mobile terminal of the waiting passenger via the communication network. In this case, the waiting passenger usually stops boarding the bus 1. However, the waiting passenger may mistakenly try to board the bus 1. Assuming such a case, in the embodiment according to the present disclosure, a speaker that conveys an instruction relating to the boarding to the bus 1 to the waiting passenger of the bus 1 by voice, or a display that notifies the waiting passenger of the bus 1 of the instruction relating to the boarding to the bus 1 by video is installed at the bus stop 50. The suspected infected person is notified of the message that boarding of the suspected infected person to the bus 1 is refused using the speaker or the display. Alternatively, a speaker that conveys the instruction relating to the boarding to the bus 1 to the waiting passenger of the bus 1 by voice, or a display that notifies the waiting passenger of the bus 1 of the instruction relating to the boarding to the bus 1 by video is installed in the bus 1, and the suspected infected person is notified of the message that boarding of the suspected infected person to the bus 1 is refused using the speaker or the display.

That is, in the embodiment according to the present disclosure, a boarding refusal device that refuses boarding of the suspected infected person when the infectious disease determination device 51 determines that the waiting passenger of the bus 1 is the suspected infected person is provided at the bus stop 50 or in the bus 1. In this case, in the embodiment according to the present disclosure, as described above, the speaker that conveys the instruction relating to the boarding to the bus 1 to the waiting passenger of the bus 1 by voice, or the display that notifies the waiting passenger of the bus 1 of the instruction relating to the boarding to the bus 1 by video is installed at least one of at the bus stop 50 and in the bus 1. The boarding refusal device notifies the suspected infected person of the message that boarding of the suspected infected person to the bus 1 is refused using at least one of the speaker and the display. In this case, when a driver, conductor, or the like is on the bus 1, it is also possible to notify the suspected infected person that the boarding of the suspected infected person to the bus 1 is refused by the driver, conductor, etc. by informing the driver, conductor, etc. that the passenger is the suspected infected person.

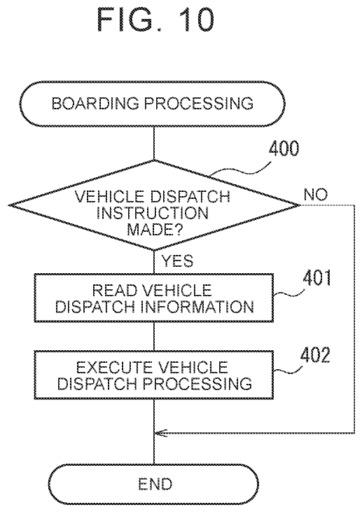

FIG. 10 shows a vehicle dispatch processing routine executed by the electronic control unit 62 provided in the vehicle dispatch coordination device 61 of the vehicle dispatch coordination department 60. With reference to FIG. 10, in step 400, the electronic control unit 62 determines whether a vehicle dispatch instruction is made to dispatch the vehicle to the bus stop 50 where the waiting passenger is present. When the vehicle dispatch instruction is not made, the processing cycle is terminated. On the other hand, when the vehicle dispatch instruction is made, the process proceeds to step 401, and the vehicle dispatch instruction information that has already been transmitted, that is, the information relating to the location of the bus stop 50 where the waiting passenger is present, the information relating to the mobile terminal of the waiting passenger, for example, the owner information of the mobile terminal, the information relating to the determination result by the infectious disease detection sensor 58, and the like are read. Next, in step 402, the vehicle dispatch processing for dispatching the dedicated vehicle 68 for the suspected infected person to the bus stop 50 where the waiting passengers is present is executed.

Next, the vehicle dispatch processing executed in step 402 will be described. In the embodiment according to the present disclosure, at this time, the dedicated vehicle 68 for the suspected infected person dispatched by the vehicle dispatch coordination department 60 is a single-seater vehicle. In this case, an autonomous driving vehicle can also be used as the dedicated vehicle 68 for the suspected infected person. Further, in the embodiment according to the present disclosure, a usage history of the dedicated vehicle 68 for the suspected infected person is stored in the memory 65 of the electronic control unit 62 of the vehicle dispatch coordination department 60. When the vehicle dispatch instruction is made, the dedicated vehicle 68 for the suspected infected person with which a preset time has elapsed since the last time the dedicated vehicle 68 for the suspected infected person is used by the suspected infected person is dispatched.

Further, in another embodiment according to the present disclosure, a disinfection history of the dedicated vehicle 68 for the suspected infected person is stored in the memory 65 of the electronic control unit 62 of the vehicle dispatch coordination department 60. When the vehicle dispatch instruction is made, the dedicated vehicle 68 for the suspected infected person that has been disinfected after the last time the dedicated vehicle 68 for the suspected infected person is used by the suspected infected person is dispatched.

What is claimed is:

1. An infection control system, comprising a vehicle dispatch coordination department that dispatches a dedicated vehicle for a suspected infected person separately from a bus when an infectious disease determination device that is installed at a bus stop and that determines whether a waiting passenger of the bus is the suspected infected person who is suspected of being infected with an infectious disease determines that the waiting passenger of the bus is the suspected infected person.

2. The infection control system according to claim 1, wherein the infectious disease determination device includes an infectious disease detection sensor for detecting whether the waiting passenger of the bus is suspected to be infected with the infectious disease, the infectious disease detection sensor being installed at the bus stop, and determines whether the waiting passenger of the bus is the suspected infected person who is suspected of being infected with the infectious disease based on a detection result detected by the infectious disease detection sensor.

3. The infection control system according to claim 2, wherein the infectious disease detection sensor includes at least one of a sensor by which a body temperature of the waiting passenger of the bus is detectable, a microphone by which a coughing sound of the waiting passenger of the bus is detectable, and a sensor by which the infectious disease is chemically detectable.

4. The infection control system according to claim 1, further comprising a boarding refusal device that refuses boarding of the suspected infected person to the bus when the infectious disease determination device determines that the waiting passenger of the bus is the suspected infected person.

5. The infection control system according to claim 4, wherein a speaker that conveys an instruction relating to the boarding to the bus to the waiting passenger of the bus by voice, or a display that notifies the waiting passenger of the bus of the instruction relating to the boarding of the bus by video is installed at least one of at the bus stop or in the bus, and the boarding refusal device notifies the suspected infected person of a message that the boarding of the suspected infected person to the bus is refused using at least one of the speaker and the display.

6. The infection control system according to claim 4, wherein a communication device for transmitting an instruction relating to the boarding to the bus to a mobile terminal of the waiting passenger of the bus is provided, and the boarding refusal device transmits a message that the boarding of the suspected infected person to the bus is refused to the mobile terminal of the suspected infected person using the communication device.

7. The infection control system according to claim 1, wherein the dedicated vehicle for the suspected infected person dispatched by the vehicle dispatch coordination department is a single-seater vehicle.

8. The infection control system according to claim 1, wherein the vehicle dispatch coordination department stores a usage history of the dedicated vehicle for the suspected infected person, and dispatches the dedicated vehicle for the suspected infected person with which a preset time has elapsed since the last time the dedicated vehicle for the suspected infected person is used by the suspected infected person.

9. The infection control system according to claim 1, wherein the vehicle dispatch coordination department stores a usage history of the dedicated vehicle for the suspected infected person, and dispatches the dedicated vehicle for the suspected infected person that has been disinfected after the last time the dedicated vehicle for the suspected infected person is used by the suspected infected person.

10. An infection control method, comprising dispatching a dedicated vehicle for a suspected infected person separately from a bus when an infectious disease determination device that is installed at a bus stop and that determines whether a waiting passenger of the bus is the suspected infected person who is suspected of being infected with an infectious disease determines that the waiting passenger of the bus is the suspected infected person.
